(19) United States
(12) Patent Application Publication
(10) Pub. No.: US 2020/0270575 A1
(43) Pub. Date: Aug. 27, 2020

(54) Culture System for Expanding Hematopoietic Stem Cells and/or Hematopoietic Progenitor Cells and the Method Thereof, Hematopoietic Stem Cells and Hematopoietic Progenitor Cells

(71) Applicants: NEWISH TECHNOLOGY (BEIJING) CO., LTD., Beijing (CN); BEIJING XIEKE MEDICAL TECHNOLOGY CO., LTD., Chaoyang, Beijing (CN)

(72) Inventors: Zhongjie Sun, Chaoyang, Beijing (CN); Xiao Guo, Chaoyang, Beijing (CN); Defang Liu, Chaoyang, Beijing (CN); Huan Yang, Chaoyang, Beijing (CN); Hongjiao Liu, Chaoyang, Beijing (CN); Yingxia Li, Chaoyang, Beijing (CN)

(73) Assignees: NEWISH TECHNOLOGY (BEIJING) CO., LTD., Beijing (CN); BEIJING XIEKE MEDICAL TECHNOLOGY CO., LTD., Chaoyang, Beijing (CN); BEIJING XIEKE MEDICAL TECHNOLOGY CO., LTD., Chaoyang, Beijing (CN)

(21) Appl. No.: 16/345,116

(22) PCT Filed: Jan. 17, 2018
(86) PCT No.: PCT/CN2018/072961

§ 371 Date: Apr. 25, 2019

(30) Foreign Application Priority Data
Dec. 29, 2017 (CN) 201711486197.4

(51) Int. Cl. C12N 5/0789

(52) U.S. Cl. CPC C12N 5/0647; C12N 2501/727

(57) ABSTRACT

The present application provides a culture system and method for expanding hematopoietic stem cells and/or hematopoietic progenitor cells, hematopoietic stem cells and hematopoietic progenitor cells. The culture system comprises a basal medium suitable for expanding stem cells; and a JNK signaling pathway inhibitor. Groups of JNK signaling pathway inhibitor are described and provided.

### TECHNICAL FIELD

The present application mainly relates to the field of biotechnology and medicine, and in particular to a culture system for expanding hematopoietic stem cells and/or hematopoietic progenitor cells and the method thereof, hematopoietic stem cells and hematopoietic progenitor cells.

### BACKGROUND

Hematopoietic stem cells are a kind of blood stem cells that have the ability to self-renew and multi-lineage differentiate, and are capable of long-term reconstituting hematopoiesis(for at least 4 months) in vivo. Compared with hematopoietic stem cells, hematopoietic progenitor cells have weaker reconstitution capacity and cannot maintain the level of reconstitution in vivo for a long time, and thus will gradually lose their reconstructed cells in 2 months in general. In 1961, scientists first demonstrated the existence of hematopoietic stem cells by a method of spleen nodules in mice. Later, the understanding of hematopoietic stem cells have been broadened through researches and functional verifications of surface marker proteins of hematopoietic stem cells by several laboratories such as Weissman of the United States. Currently, it can be determined preliminarily that hematopoietic stem and progenitor cells (i.e. hematopoietic stem cells and hematopoietic progenitor cells) are enriched in CD34+CD45RA− surface protein-labeled cells. And CD34+CD38−CD90+CD45RA−CD49f+ labeled cells are considered as hematopoietic stem cells having the ability of long-term hematopoietic reconstitution (LT-HSC).

Clinically, the most effective method for treating blood diseases such as leukemia is transplanting hematopoietic stem cells, but there are reasons such as the difficulty in bone marrow matching that lead to nearly 40% of patients failing to find a match. Although hematopoietic stem cells in cord blood have many advantages such as low immunogenicity, the limited number of hematopoietic stem cells in cord blood limits their clinical application. Therefore, how to efficiently expand cord blood-derived hematopoietic stem cells in vitro has great scientific value and clinical application value.

Jun N-terminal kinase (JNK) family is one of members of mitogen-activated protein kinase (MAPK) super family. The genes encoding JNK currently found in mammalian cells include jnk1, jnk2 and jnk3, and their corresponding coding products JNK1 and JNK2 are widely expressed, and JNK3 is mainly expressed in the nervous system. The JNK signaling pathway centered on JNK is an important branch of the MAPK (mitogen-activated protein kinase) pathway. After being discovered in 1991, it has attracted wide attention from scientists, however, when compared to P38 and ERK, the other two subtypes of MAPK pathway, it is less studied by the scientists. JNK signaling pathway plays a vital role in cell regulation, such as cell differentiation, apoptosis, and stress response. A large number of experiments have confirmed that it is closely related to many diseases. Therefore, inhibition of JNK signaling pathway has become a means for treating many diseases. JNK signaling pathway inhibitors are mainly used in studies of various cancer cells. It is reported that JNK inhibitors can inhibit the proliferation of bladder cancer and lung cancer cells in a mouse model and tumor atrophy can be observed obviously. JNK signaling pathway inhibitors also play an important role in regulating the self-renewal and differentiation of pluripotent stem cells. In 2014, it was reported by Nature magazine that Jacob Hanna Laboratory has used a combination of small chemical molecules (including the JNK inhibitor SP600125) to keep pluripotent stem cells in a more naive state and thus improve the chimeric capacity of embryos. However, at present, studies on the expanding of human hematopoietic stem cells by using JNK small molecule inhibitors have not been reported.

Small molecule compounds are a kind of natural molecular products or synthetic compounds with a molecular weight less than 1000 daltons, which have certain biological functions. Small molecules have attracted much attention in clinical research because of their advantages such as high cell permeability, simple synthesis, and no damage to the cell genome. In 2010, the purine derivative compound Stem Regenin (SR1) was the first small molecule being discovered that can largely expand the hematopoietic stem and progenitor cells in vitro. SR1 can increase CD34+ cells by inhibiting the AHR signaling pathway, and the amount of exogenous hematopoietic cells detected in immunodeficiency mice was increased by 17 times. In 2014, the Canadian research team found that the pyrimidine molecule UM171 can effectively increase LT-HSC and maintain the function of hematopoietic reconstitution for up to 6 months through a small-molecule high-throughput screening platform, which widens the clinical application value and scope of cord blood hematopoietic stem cells. However, the small molecule compounds have a wide range of targets, and their signaling pathways are not unitary, which may limit the scales of their clinical application.

### SUMMARY

It is an object of the present application to provide a culture system for expanding hematopoietic stem cells and/or hematopoietic progenitor cells and the method thereof, hematopoietic stem cells, and hematopoietic progenitor cells.

The present application provides a culture system for expanding hematopoietic stem cells and/or hematopoietic progenitor cells, comprising a basal medium suitable for expanding stem cells; and a JNK signaling pathway inhibitor. Preferably, the INK signaling pathway inhibitor is a small molecule compound.

Preferably, according to the aforementioned culture system, wherein the INK signaling pathway inhibitor is selected from one or more of the group consisting of JNK1 inhibitors, JNK2 inhibitors and JNK3 inhibitors.

Preferably, according to the aforementioned culture system, wherein the INK signaling pathway inhibitor is selected from one or more of the group consisting of SP600125, AS601245 and AEG-3482.

The chemical formula of SP600125 is as follow:

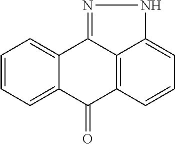 [SMILES: O=C1c2ccccc2-c2n[nH]c3cccc1c23]

The chemical formula of AS601245 is as follow:

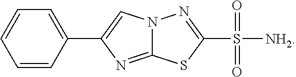 [SMILES: NS(=O)(=O)c1nn2cc(-c3ccccc3)nc2s1]

The chemical formula of AEG-3482 is as follow:

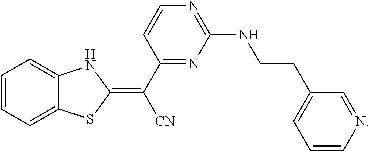 [SMILES: N#C/C(=C1/Nc2ccccc2S1)c1ccnc(NCCc2cccnc2)n1]

Or preferably, according to the aforementioned culture system, wherein the JNK signaling pathway inhibitor is any of the compounds represented by JNK-IN-1 to INK-IN-12 as follows:

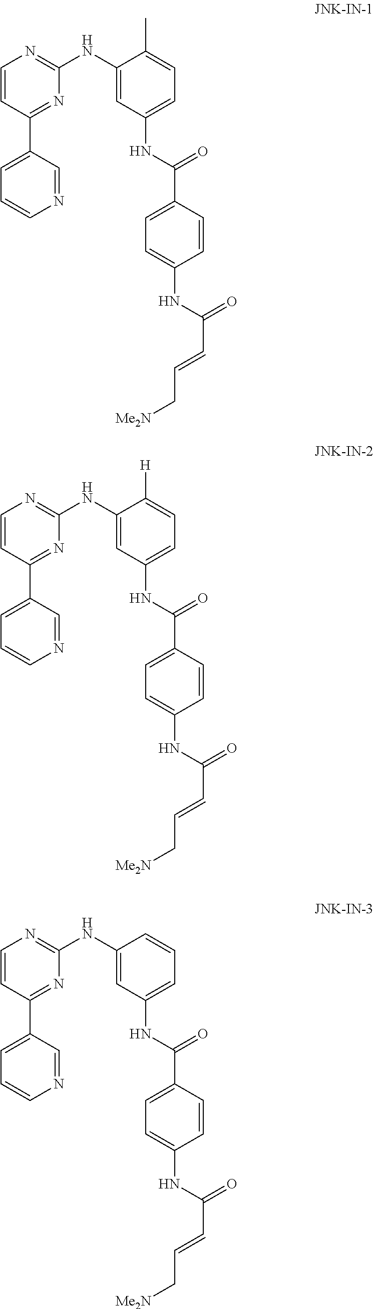 [SMILES: Cc1ccc(NC(=O)c2ccc(NC(=O)/C=C/CN(C)C)cc2)cc1Nc1nccc(-c2cccnc2)n1 ; CN(C)C/C=C/C(=O)Nc1ccc(C(=O)Nc2cccc(Nc3nccc(-c4cccnc4)n3)c2)cc1 ; CN(C)C/C=C/C(=O)Nc1ccc(C(=O)Nc2cccc(Nc3nccc(-c4cccnc4)n3)c2)cc1]

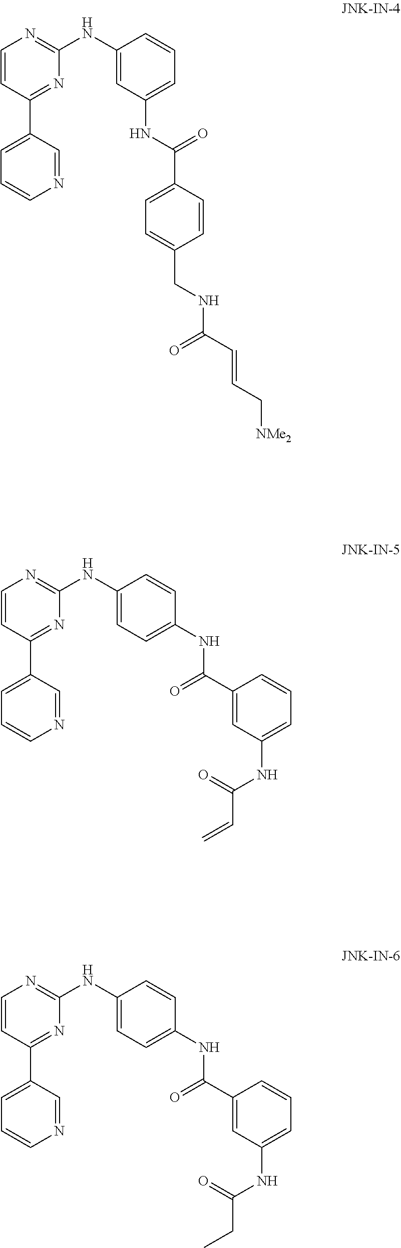 [SMILES: CN(C)C/C=C/C(=O)NCc1ccc(C(=O)Nc2cccc(Nc3nccc(-c4cccnc4)n3)c2)cc1 ; C=CC(=O)Nc1cccc(C(=O)Nc2ccc(Nc3nccc(-c4cccnc4)n3)cc2)c1 ; CCC(=O)Nc1cccc(C(=O)Nc2ccc(Nc3nccc(-c4cccnc4)n3)cc2)c1]

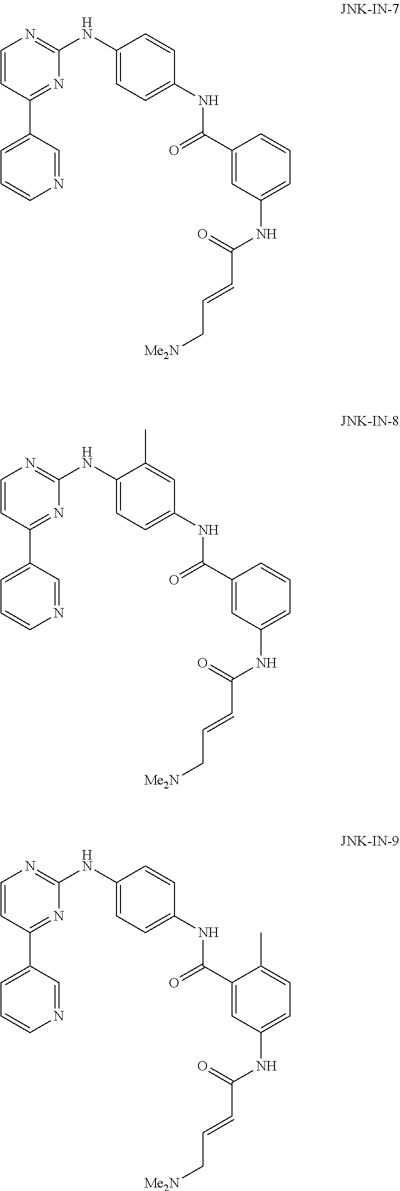 [SMILES: CN(C)C/C=C/C(=O)Nc1cccc(C(=O)Nc2ccc(Nc3nccc(-c4cccnc4)n3)cc2)c1 ; Cc1cc(NC(=O)c2cccc(NC(=O)/C=C/CN(C)C)c2)ccc1Nc1nccc(-c2cccnc2)n1 ; Cc1ccc(NC(=O)/C=C/CN(C)C)cc1C(=O)Nc1ccc(Nc2nccc(-c3cccnc3)n2)cc1]

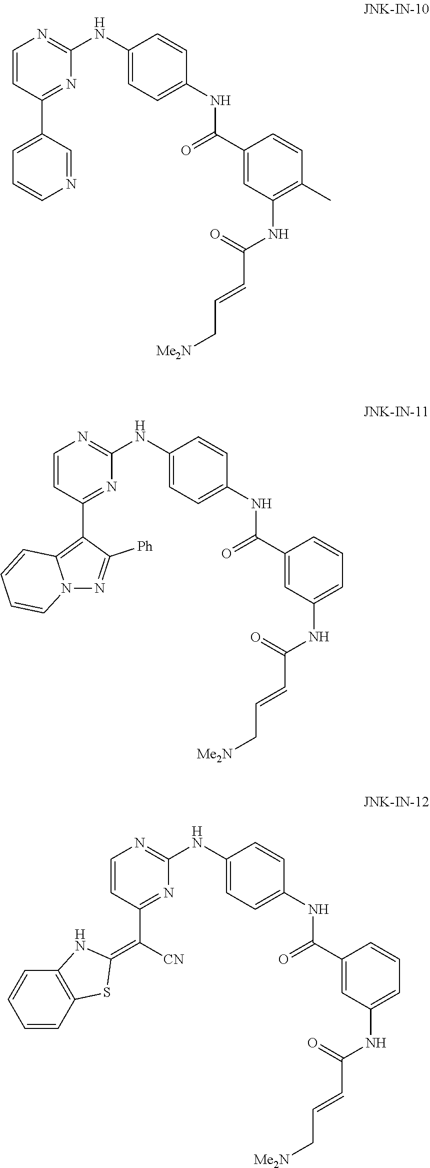 [SMILES: Cc1ccc(C(=O)Nc2ccc(Nc3nccc(-c4cccnc4)n3)cc2)cc1NC(=O)/C=C/CN(C)C ; CN(C)C/C=C/C(=O)Nc1cccc(C(=O)Nc2ccc(Nc3nccc(-c4c(-c5ccccc5)nn5ccccc45)n3)cc2)c1 ; CN(C)C/C=C/C(=O)Nc1cccc(C(=O)Nc2ccc(Nc3nccc(/C(C#N)=C4\Nc5ccccc5S4)n3)cc2)c1]

The above INK signaling pathway inhibitors were constructed according to Tinghu Zhang et al. (Chem Biol. 2012 Jan. 27; 19(1): 140-154).

More preferably, according to the aforementioned culture system, wherein the basal medium includes StemSpan basal medium 1-100 ml; recombinant human stem cell factor (SCF) 10-500 ng/ml; Flt3 ligand (FL) 10-100 ng/ml, preferably 50-100 ng/ml; recombinant human thrombopoietin (TPO) 5-100 ng/ml, preferably 5-50 ng/ml; low-density lipoprotein (LDL) 2-10 μg/ml; and the JNK signaling pathway inhibitor is 0.5-10 82 M, preferably 0.5-5 μM.

The present application also provides a method for expanding hematopoietic stem cells and/or hematopoietic progenitor cells, wherein hematopoietic stem cells and/or hematopoietic progenitor cells are cultured in vitro with the presence of the JNK signaling pathway inhibitor.

Preferably, according to the aforementioned method, wherein the JNK signaling pathway inhibitor is selected from one or more of the group consisting of JNK1 inhibitors, JNK2 inhibitors and JNK3 inhibitors.

Preferably, according to the aforementioned method, wherein the JNK signaling pathway inhibitor is selected from one or more of the group consisting of SP600125, AS601245 and AEG-3482.

Preferably, according to the aforementioned method, wherein the JNK signaling pathway inhibitor is any of the compounds represented by JNK-IN-1 to JNK-IN-12 as follows:

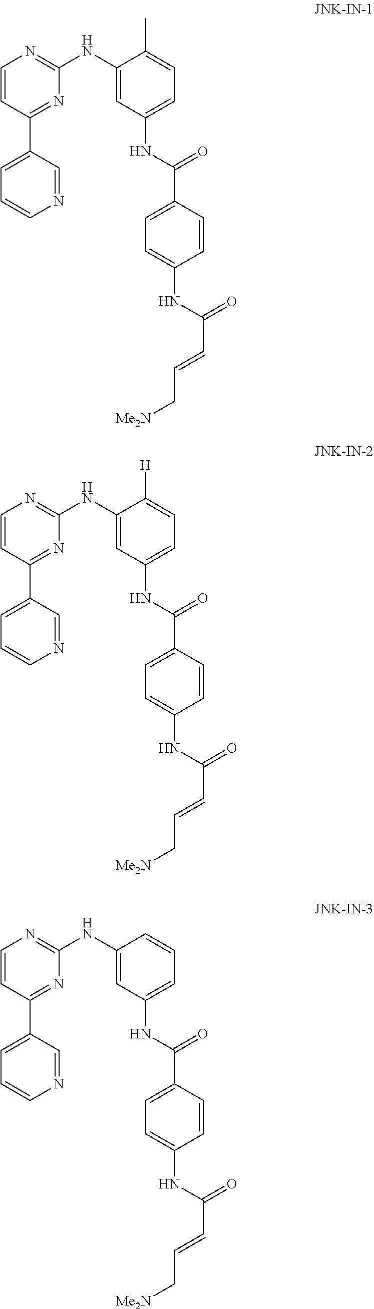 [SMILES: Cc1ccc(NC(=O)c2ccc(NC(=O)/C=C/CN(C)C)cc2)cc1Nc1nccc(-c2cccnc2)n1 ; CN(C)C/C=C/C(=O)Nc1ccc(C(=O)Nc2cccc(Nc3nccc(-c4cccnc4)n3)c2)cc1 ; CN(C)C/C=C/C(=O)Nc1ccc(C(=O)Nc2cccc(Nc3nccc(-c4cccnc4)n3)c2)cc1]

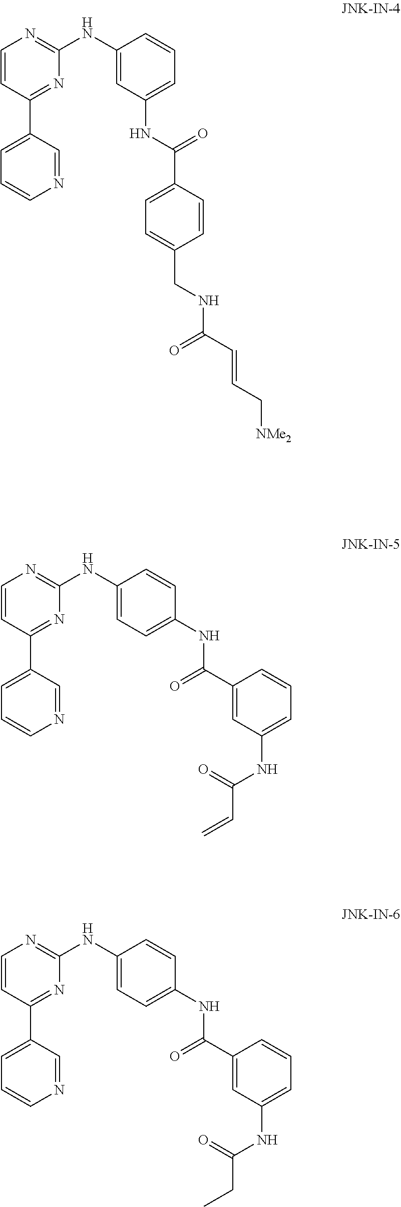 [SMILES: CN(C)C/C=C/C(=O)NCc1ccc(C(=O)Nc2cccc(Nc3nccc(-c4cccnc4)n3)c2)cc1 ; C=CC(=O)Nc1cccc(C(=O)Nc2ccc(Nc3nccc(-c4cccnc4)n3)cc2)c1 ; CCC(=O)Nc1cccc(C(=O)Nc2ccc(Nc3nccc(-c4cccnc4)n3)cc2)c1]

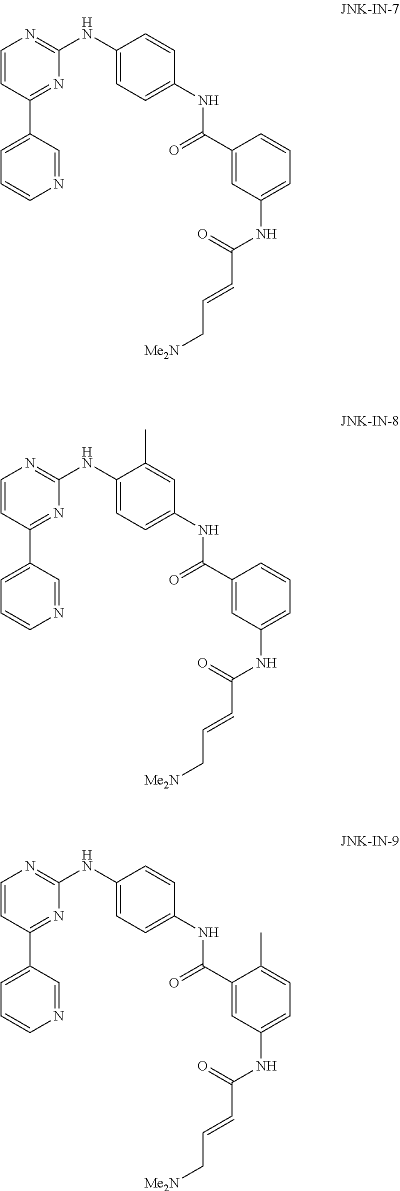 [SMILES: CN(C)C/C=C/C(=O)Nc1cccc(C(=O)Nc2ccc(Nc3nccc(-c4cccnc4)n3)cc2)c1 ; Cc1cc(NC(=O)c2cccc(NC(=O)/C=C/CN(C)C)c2)ccc1Nc1nccc(-c2cccnc2)n1 ; Cc1ccc(NC(=O)/C=C/CN(C)C)cc1C(=O)Nc1ccc(Nc2nccc(-c3cccnc3)n2)cc1]

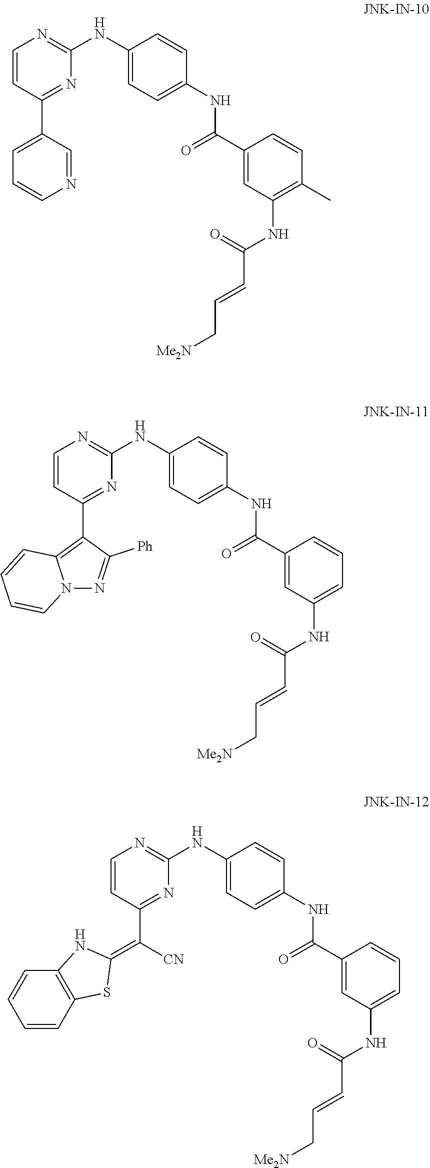 [SMILES: Cc1ccc(C(=O)Nc2ccc(Nc3nccc(-c4cccnc4)n3)cc2)cc1NC(=O)/C=C/CN(C)C ; CN(C)C/C=C/C(=O)Nc1cccc(C(=O)Nc2ccc(Nc3nccc(-c4c(-c5ccccc5)nn5ccccc45)n3)cc2)c1 ; CN(C)C/C=C/C(=O)Nc1cccc(C(=O)Nc2ccc(Nc3nccc(/C(C#N)=C4\Nc5ccccc5S4)n3)cc2)c1]

Or preferably, according to the aforementioned method, wherein the method comprises the addition of StemSpan basal medium, recombinant human stem cell factor, Flt3 ligand, recombinant human thrombopoietin and low-density lipoprotein.

More preferably, according to the aforementioned culture system and aforementioned method, wherein the hematopoietic stem cells and/or hematopoietic progenitor cells are derived from bone marrow, liver, spleen, peripheral blood or cord blood.

The present application also provides hematopoietic stem cells, wherein the hematopoietic stem cells are expanded by the above culture system or the above method. Specifically, the CD34+ cells derived from cord blood are expanded by the above culture system or the above method to prepare the hematopoietic stem cells.

The present application also provides hematopoietic progenitor cells, wherein the hematopoietic progenitor cells are expanded by the above culture system or the above method. Specifically, the CD34+ cells derived from cord blood are expanded by the above culture system or the above method to prepare the hematopoietic progenitor cells.

The culture system provided by the present application can expand hematopoietic stem cells in vitro obviously, and the CD34+CD45RA−labeled hematopoietic stem cells can be expanded by nearly 100 times.

The culture system and the method thereof provided by the present invention use small chemical molecules which have clear targets, and is safer in clinical use.

The hematopoietic stem cells and/or hematopoietic progenitor cells expanded by the culture system or the method provided by the present invention have greater reconstruction capacity in vivo, and the reconstruction effect in vivo is improved by 10 times when compared with the experimental group without the small molecule compound.

### BRIEF DESCRIPTION OF DRAWINGS

FIG. 1 shows the fold expansion of the detected CD34+CD45RA− cells at day 7 relative to the primary cells in Example 1.

FIG. 2 shows the reconstitution proportion of bone marrow of the transplantation experiment in vivo in Example 1.

FIG. 3 shows the reconstitution proportion of peripheral blood of the transplantation experiment in vivo in Example 1.

FIG. 4 shows the fold expansion of the detected CD34+CD45RA− cells at day 7 relative to the primary cells in Example 2.

FIG. 5 shows the reconstitution proportion of bone marrow of the transplantation experiment in vivo in Example 2.

FIG. 6 shows the reconstitution proportion of peripheral blood of the transplantation experiment in vivo in Example 2.

FIG. 7 shows the fold expansion of the detected CD34+CD45RA− cells at day 7 relative to the primary cells in Example 3.

FIG. 8 shows the reconstitution proportion of bone marrow of the transplantation experiment in vivo in Example 3.

FIG. 9 shows the reconstitution proportion of peripheral blood of the transplantation experiment in vivo in Example 3.

FIG. 10 shows the fold expansion of the detected CD34+CD45RA− cells at day 7 relative to the primary cells in Example 4.

FIG. 11 shows the reconstitution proportion of bone marrow of the transplantation experiment in vivo in Example 4.

FIG. 12 shows the reconstitution proportion of peripheral blood of the transplantation experiment in vivo in Example 4.

FIG. 13 shows the fold expansion of the detected CD34+CD45RA− cells at day 7 relative to the primary cells in Example 5.

FIG. 14 shows the reconstitution proportion of bone marrow of the transplantation experiment in vivo in Example 5.

FIG. 15 shows the reconstitution proportion of peripheral blood of the transplantation experiment in vivo in Example 5.

FIG. 16 shows the fold expansion of the detected CD34+CD45RA− cells at day 7 relative to the primary cells in Example 6.

FIG. 17 shows the reconstitution proportion of bone marrow of the transplantation experiment in vivo in Example 6.

FIG. 18 shows the reconstitution proportion of peripheral blood of the transplantation experiment in vivo in Example 6.

FIG. 19 shows the fold expansion of the detected CD34+CD45RA− cells at day 7 relative to the primary cells in Example 7.

FIG. 20 shows the reconstitution proportion of bone marrow of the transplantation experiment in vivo in Example 7.

FIG. 21 shows the reconstitution proportion of peripheral blood of the transplantation experiment in vivo in Example 7.

### Reference Marks

DMSO: the control group; Y1: the experimental group.

### DETAILED DESCRIPTION

In order to describe the specific implementation modalities of the present invention in more details, the following examples and figures are provided in order to better understand the solution of the present invention and its advantages of various aspects. However, the specific implementation modalities and examples described below are illustrative only and not limiting to the invention.

For ease of understanding, the present invention will be described in detail below through specific implementation modalities. It needs to be noted that these descriptions are merely exemplary description and does not limit the scope of the present invention. According to the present descriptions, many variations and modifications of the invention are obvious for those skilled in the field.

Further, the present invention cited references to more clearly describe the present invention. The entire contents thereof are incorporated herein for reference as if they had been repeated as described herein.

The contents of the present invention are further illustrated by the following examples. Unless otherwise specified, the technical means used in the examples are conventional means well known to those skilled in the field and commercially available instruments and reagents. Reference can be made to Molecular Cloning : A Laboratory Manual (3rd Edition) (Science Press), Microbiology Experiments (4th Edition) (Higher Education Press) and manufacturer's instructions for the corresponding instruments and reagents.

The StemSpan basal medium is a basal medium produced by STEMCELL.

Small chemical molecules are prepared by many biological reagent companies such as Tocris and Selleck.

The material was derived from cord blood waste supplied by the company, and cord blood-derived CD34+ cells were isolated by magnetic bead sorting (MACS) as cultured materials in the following examples.

TABLE 1
medium composition and concentration of the examples

| | Concentration | | | | | | |
|---|---|---|---|---|---|---|---|
| | Ex- | Ex- | Ex- | Ex- | Ex- | Ex- | Ex- |
| Medium | ample | ample | ample | ample | ample | ample | ample |
| composition | 1 | 2 | 3 | 4 | 5 | 6 | 7 |
| stemspan (ml) | 100 | 100 | 100 | 100 | 100 | 100 | 100 |
| SCF (ng/ml) | 100 | 50 | 20 | 10 | 200 | 300 | 500 |
| FL (ng/ml) | 100 | 50 | 50 | 100 | 50 | 100 | 100 |
| TPO (ng/ml) | 50 | 25 | 20 | 10 | 5 | 20 | 20 |
| LDL (μg/ml) | 10 | 5 | 5 | 2 | 10 | 10 | 10 |
| Small chemical | 4 | 4 | 2 | 5 | 1 | 0.5 | 5 |
| molecules (μM) | | | | | | | |

### Example 1

In this example, CD34+ cells were cultured using the culture system provided by the present invention.

CD34+ cells were cultured based on the culture conditions of Table 1. The experimental group (the small chemical molecule is SP600125) and the control group (the small chemical molecule is replaced by DMSO, the volume ratio of DMSO: medium is 1:10000) were placed in the cell incubator at 37° C. with the concentration of 5% carbon dioxide, and half amount of the medium was changed every 2 days.

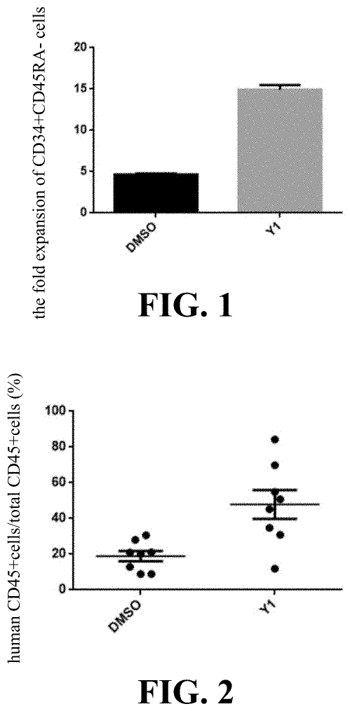

On the 7th day of the culturing, the ratio of CD34+CD45RA−cells was measured, and the fold expansion was counted and calculated. The results are shown in FIG. 1. The CD34+CD45RA− in the experimental group was about 15 times in the fold expansion, while the CD34+CD45RA− in the control group was about 5 times in fold expansion.

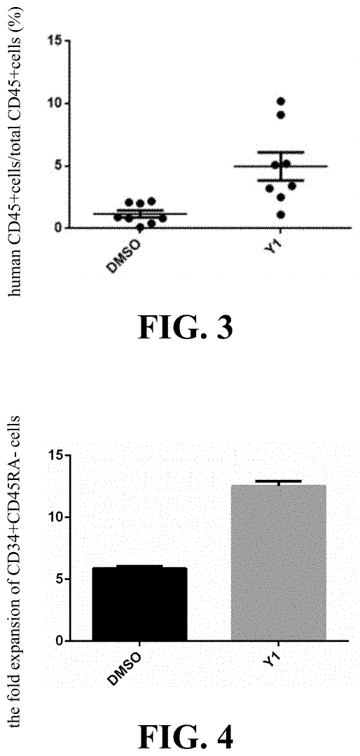

On the 7th day of the culturing, the transplantation experiment in vivo was carried out at the meantime. The expanded CD34+ cells in the experimental group and the control group were respectively injected into the immunodeficient mice by bone marrow transplantation. Each group contain 8 mice, and each mouse was injected with the total cells expanded from 5000 original CD34+ cells. The reconstitution ratio of human cells in peripheral blood and bone marrow of immunodeficient mice were measured after 4 months. The results are shown in FIGS. 2 and 3. In the experimental group, the reconstruction proportion of human blood cells in bone marrow (CD45+ cells) reached about 50%, and the blood cells in human peripheral blood reached about 5%. In the control group, the reconstruction proportion of human blood cells in bone marrow reached about 20%, and the blood cells in human peripheral blood reached about 1%. The reconstruction effect of the experimental group was about 4 times higher than that of the control group after a general comparison.

The above experiments have proved that the culture system provided by the invention can efficiently expand hematopoietic stem cells, and the hematopoietic stem cells cultured in vitro by the culture system have better reconstruction capacity in vivo.

### Example 2

In this example, CD34+ cells were cultured using the culture system provided by the present invention.

CD34+ cells were cultured based on the culture conditions of Table 1. The experimental group (the small chemical molecule is SP600125) and the control group (the small chemical molecule is replaced by DMSO, the volume ratio of DMSO: medium is 1:10000) were placed in the cell incubator at 37° C. with the concentration of 5% carbon dioxide, and half amount of the medium was changed every 2 days.

On the 7th day of the culturing, the ratio of CD34+CD45RA−cells was measured, and the fold expansion was counted and calculated. The results are shown in FIG. 4. The CD34+CD45RA− in the experimental group was 2.5 times higher than that in the control group.

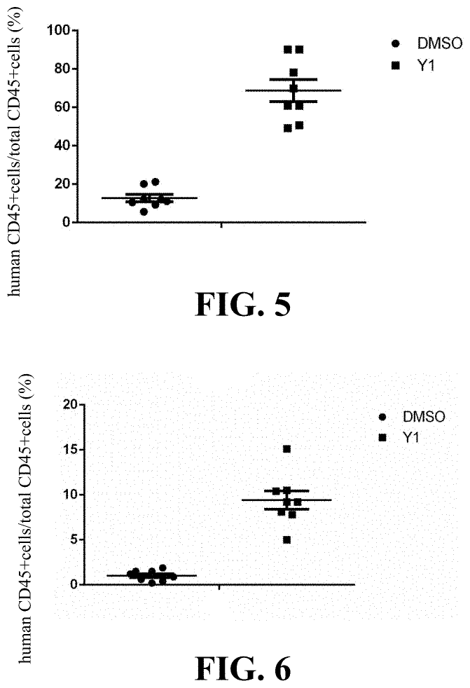

On the 7th day of the culturing, the transplantation experiment in vivo was carried out at the meantime. The expanded CD34+ cells in the experimental group and the control group were respectively injected into the immunodeficient mice by bone marrow transplantation. Each group contain 8 mice, and each mouse was injected with the total cells expanded from 5000 original CD34+ cells. The reconstitution ratio of human cells in peripheral blood and bone marrow of immunodeficient mice were measured after 4 months. The results are shown in FIGS. 5 and 6. In the experimental group, the reconstruction proportion of human blood cells in bone marrow (CD45+ cells) reached about 70%, and the blood cells in human peripheral blood reached about 9%. In the control group, the reconstruction proportion of human blood cells in bone marrow reached about 15%, and the blood cells in human peripheral blood reached about 1%. The reconstruction effect of the experimental group was about 6 times higher than that of the control group after a general comprehensive comparison.

The above experiments have proved that the culture system provided by the invention can efficiently expand hematopoietic stem cells, and the hematopoietic stem cells cultured in vitro by the culture system have better reconstruction capacity in vivo.

### Example 3

In this example, CD34+ cells were cultured using the culture system provided by the present invention.

CD34+ cells were cultured based on the culture conditions of Table 1. The experimental group (the small chemical molecule is SP600125) and the control group (the small chemical molecule is replaced by DMSO, the volume ratio of DMSO: medium is 1:10000) were placed in the cell incubator at 37° C. with the concentration of 5% carbon dioxide, and half amount of the medium was changed every 2 days.

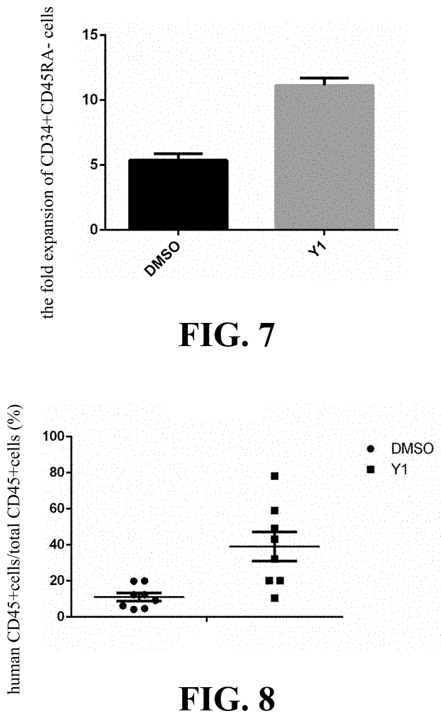

On the 7th day of the culturing, the ratio of CD34+CD45RA−cells was measured, and the fold expansion was counted and calculated. The results are shown in FIG. 7. The proliferation amount of CD34+CD45RA− in the experimental group was 2 times higher than that in the control group.

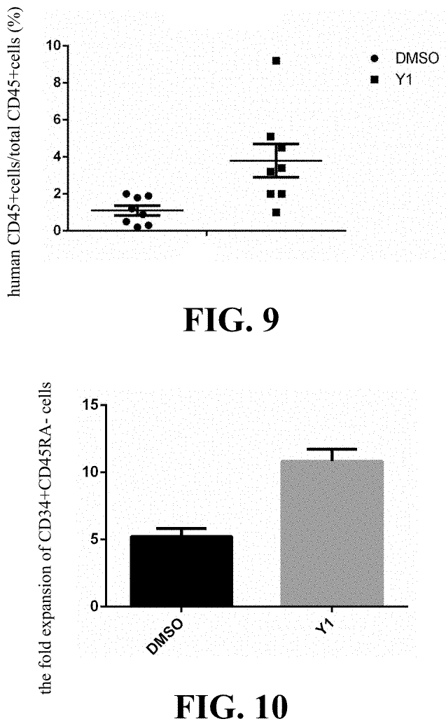

On the 7th day of the culturing, the transplantation experiment in vivo was carried out at the meantime. The expanded CD34+ cells in the experimental group and the control group were respectively injected into the immunodeficient mice by bone marrow transplantation. Each group contain 8 mice, and each mouse was injected with the total cells expanded from 5000 original CD34+ cells. The reconstitution ratio of human cells in peripheral blood and bone marrow of immunodeficient mice were measured after 4 months. The results are shown in FIGS. 8 and 9. In the experimental group, the reconstruction proportion of human blood cells in bone marrow (CD45+ cells) reached about 40%, and the blood cells in human peripheral blood reached about 4%. In the control group, the reconstruction proportion of human blood cells in bone marrow reached about 10%, and the blood cells in human peripheral blood reached about 1%. The reconstruction effect of the experimental group was about 4 times higher than that of the control group after a general comparison.

The above experiments have proved that the culture system provided by the invention can efficiently expand hematopoietic stem cells, and the hematopoietic stem cells cultured in vitro by the culture system have better reconstruction capacity in vivo.

### Example 4

In this example, CD34+ cells were cultured using the culture system provided by the present invention.

CD34+ cells were cultured based on the culture conditions of Table 1. The experimental group (the small chemical molecule is AEG-3482) and the control group (the small chemical molecule is replaced by DMSO, the volume ratio of DMSO: medium is 1:10000) were placed in the cell incubator at 37° C. with the concentration of 5% carbon dioxide, and half amount of the medium was changed every 2 days.

On the 7th day of the culturing, the ratio of CD34+CD45RA−cells was measured, and the fold expansion was counted and calculated. The results are shown in FIG. 10. The proliferation amount of CD34+CD45RA− in the experimental group was 2.3 times higher than that in the control group.

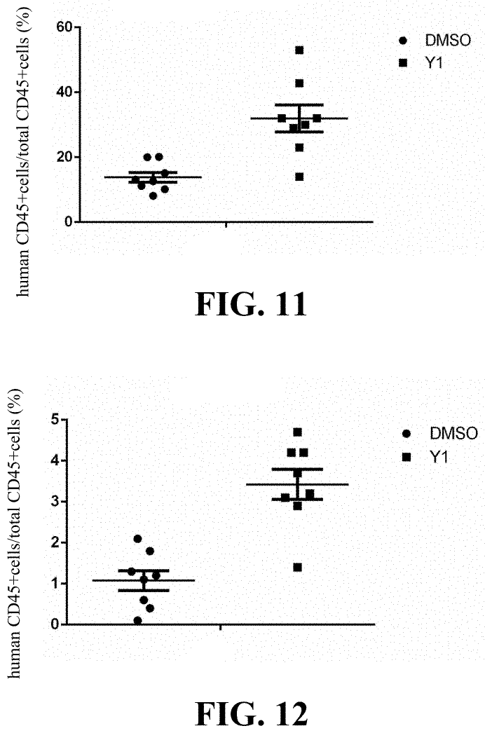

On the 7th day of the culturing, the transplantation experiment in vivo was carried out at the meantime. The expanded CD34+ cells in the experimental group and the control group were respectively injected into the immunodeficient mice by bone marrow transplantation. Each group contain 8 mice, and each mouse was injected with the total cells expanded from 5000 original CD34+ cells. The reconstitution ratio of human cells in peripheral blood and bone marrow of immunodeficient mice were measured after 4 months. The results are shown in FIGS. 11 and 12. In the experimental group, the reconstruction proportion of human blood cells in bone marrow (CD45+ cells) reached about 35%, and the blood cells in human peripheral blood reached about 3.5%. In the control group, the reconstruction proportion of human blood cells in bone marrow reached about 15%, and the blood cells in human peripheral blood reached about 1.5%. The reconstruction effect of the experimental group was about 2.5 times higher than that of the control group after a general comparison.

The above experiments have proved that the culture system provided by the invention can efficiently expand hematopoietic stem cells, and the hematopoietic stem cells cultured in vitro by the culture system have better reconstruction capacity in vivo.

### Example 5

In this example, CD34+ cells were cultured using the culture system provided by the present invention.

CD34+ cells were cultured based on the culture conditions of Table 1. The experimental group (the small chemical molecule is JNK-IN-7) and the control group (the small chemical molecule is replaced by DMSO, the volume ratio of DMSO: medium is 1:10000) were placed in the cell incubator at 37° C. with the concentration of carbon dioxide, and half amount of the medium was changed every 2 days.

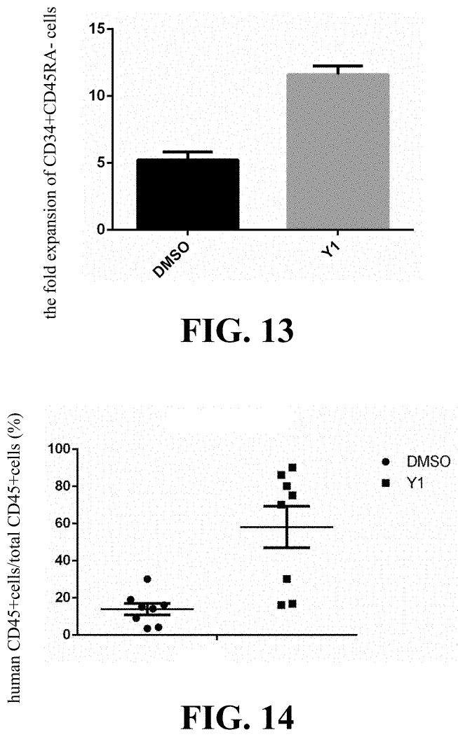

On the 7th day of the culturing, the ratio of CD34+CD45RA−cells was measured, and the fold expansion was counted and calculated. The results are shown in FIG. 13. The proliferation amount of CD34+CD45RA− in the experimental group was 2.5 times higher than that in the control group.

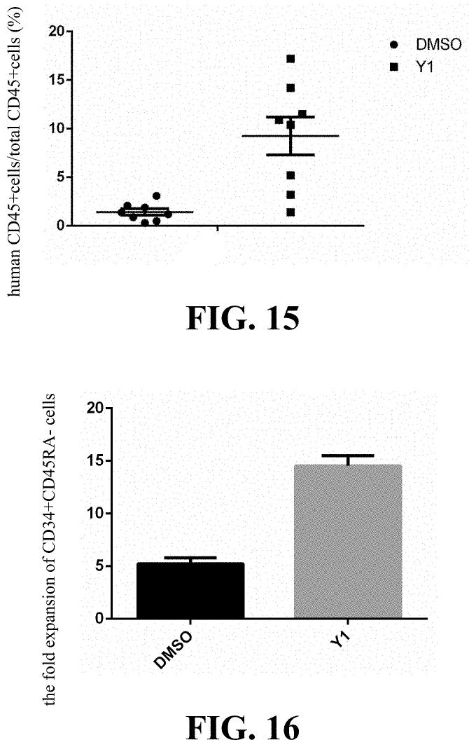

On the 7th day of the culturing, the transplantation experiment in vivo was carried out at the meantime. The expanded CD34+ cells in the experimental group and the control group were respectively injected into the immunodeficient mice by bone marrow transplantation. Each group contain 8 mice, and each mouse was injected with the total cells expanded from 5000 original CD34+ cells. The reconstitution ratio of human cells in peripheral blood and bone marrow of immunodeficient mice were measured after 4 months. The results are shown in FIGS. 14 and 15. In the experimental group, the reconstruction proportion of human blood cells in bone marrow (CD45+cells) reached about 58%, and the blood cells in human peripheral blood reached about 8.5%. In the control group, the reconstruction proportion of human blood cells in bone marrow reached about 17%, and the blood cells in human peripheral blood reached about 1.5%. The reconstruction effect of the experimental group was about 5 times higher than that of the control group after a general comparison.

The above experiments have proved that the culture system provided by the invention can efficiently expand hematopoietic stem cells, and the hematopoietic stem cells cultured in vitro by the culture system have better reconstruction capacity in vivo.

### Example 6

In this example, CD34+ cells were cultured using the culture system provided by the present invention.

CD34+ cells were cultured based on the culture conditions of Table 1. The experimental group (the small chemical molecule is JNK-IN-11) and the control group (the small chemical molecule is replaced by DMSO, the volume ratio of DMSO: medium is 1:10000) were placed in the cell incubator at 37° C. with the concentration of carbon dioxide, and half amount of the medium was changed every 2 days.

On the 7th day of the culturing, the ratio of CD34+CD45RA−cells was measured, and the fold expansion was counted and calculated. The results are shown in FIG. 16. The proliferation amount of CD34+CD45RA− in the experimental group was 2.8 times higher than that in the control group.

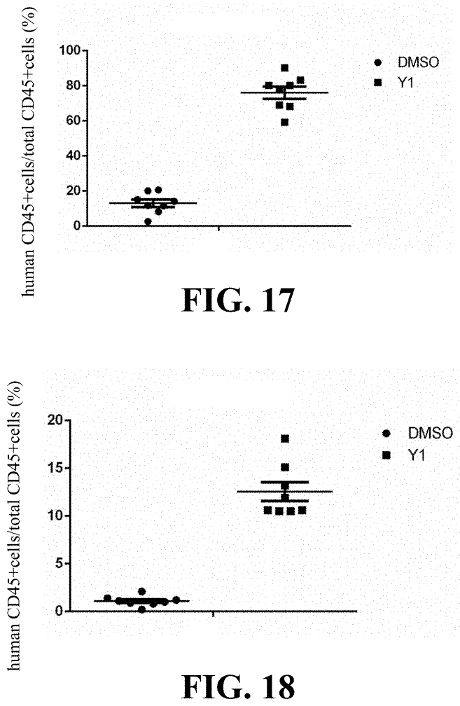

On the 7th day of the culturing, the transplantation experiment in vivo was carried out at the meantime. The expanded CD34+ cells in the experimental group and the control group were respectively injected into the immunodeficient mice by bone marrow transplantation. Each group contain 8 mice, and each mouse was injected with the total cells expanded from 5000 original CD34+ cells. The reconstitution ratio of human cells in peripheral blood and bone marrow of immunodeficient mice were measured after 4 months. The results are shown in FIGS. 17 and 18. In the experimental group, the reconstruction proportion of human blood cells in bone marrow (CD45+ cells) reached about 78%, and the blood cells in human peripheral blood reached about 12.5%. In the control group, the reconstruction proportion of human blood cells in bone marrow reached about 15%, and the blood cells in human peripheral blood reached about 1.5%. The reconstruction effect of the experimental group was about 8 times higher than that of the control group after a general comparison.

The above experiments have proved that the culture system provided by the invention can efficiently expand hematopoietic stem cells, and the hematopoietic stem cells cultured in vitro by the culture system have better reconstruction capacity in vivo.

### Example 7

In this example, CD34+ cells were cultured using the culture system provided by the present invention.

CD34+ cells were cultured based on the culture conditions of Table 1. The experimental group (the small chemical molecule is SP600125) and the control group (the small chemical molecule is replaced by DMSO, the volume ratio of DMSO: medium is 1:10000) were placed in the cell incubator at 37° C. with the concentration of carbon dioxide, and half amount of the medium was changed every 2 days.

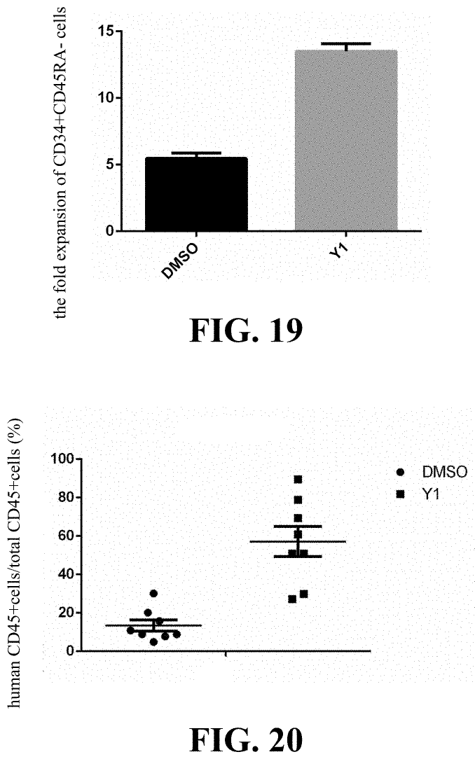

On the 7th day of the culturing, the ratio of CD34+CD45RA−cells was measured, and the fold expansion was counted and calculated. The results are shown in FIG. 19. The proliferation amount of CD34+CD45RA− in the experimental group was 2.3 times higher than that in the control group.

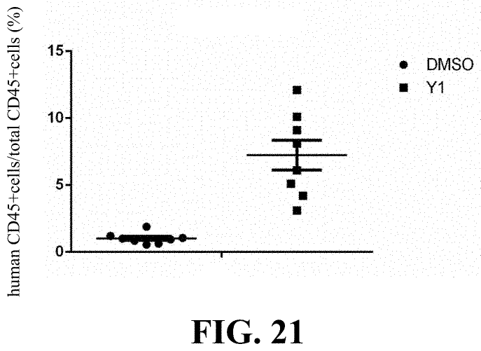

On the 7th day of the culturing, the transplantation experiment in vivo was carried out at the meantime. The expanded CD34+ cells in the experimental group and the control group were respectively injected into the immunodeficient mice by bone marrow transplantation. Each group contain 8 mice, and each mouse was injected with the total cells expanded from 5000 original CD34+ cells. The reconstitution ratio of human cells in peripheral blood and bone marrow of immunodeficient mice were measured after 4 months. The results are shown in FIGS. 20 and 21. In the experimental group, the reconstruction proportion of human blood cells in bone marrow (CD45+ cells) reached about 58%, and the blood cells in human peripheral blood reached about 7.5%. In the control group, the reconstruction proportion of human blood cells in bone marrow reached about 16%, and the blood cells in human peripheral blood reached about 1.2%. The reconstruction effect of the experimental group was about 5.5 times higher than that of the control group after a general comparison.

The above experiments have proved that the culture system provided by the invention can efficiently expand hematopoietic stem cells, and the hematopoietic stem cells cultured in vitro by the culture system have better reconstruction capacity in vivo.

It should be noted that the above-described embodiments are merely illustrative of the invention and are not intended to limit the embodiments. Other variations or modifications of the various forms may be made by those skilled in the field in light of the above description. There is no need and no way to exhaust all of the implementations. Obvious changes or variations resulting therefrom are still within the scope of the invention.

1. A culture system for expanding hematopoietic stem cells and/or hematopoietic progenitor cells, comprising a basal medium suitable for expanding stem cells; and a JNK signaling pathway inhibitor.

2. The culture system according to claim 1, wherein the JNK signaling pathway inhibitor is selected from one or more of the group consisting of JNK1 inhibitors, JNK2 inhibitors and JNK3 inhibitors.

3. The culture system according to claim 1, wherein the JNK signaling pathway inhibitor is selected from one or more of the group consisting of SP600125, AS601245 and AEG-3482.

4. The culture system according to claim 1, wherein the JNK signaling pathway inhibitor is any of the compounds represented by JNK-IN-1 to JNK-IN-12 as follows:

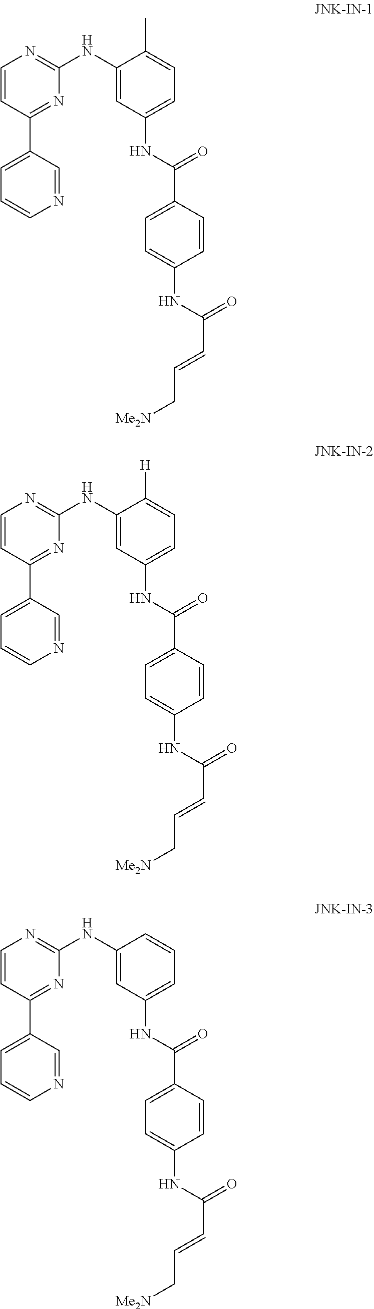 [SMILES: Cc1ccc(NC(=O)c2ccc(NC(=O)/C=C/CN(C)C)cc2)cc1Nc1nccc(-c2cccnc2)n1 ; CN(C)C/C=C/C(=O)Nc1ccc(C(=O)Nc2cccc(Nc3nccc(-c4cccnc4)n3)c2)cc1 ; CN(C)C/C=C/C(=O)Nc1ccc(C(=O)Nc2cccc(Nc3nccc(-c4cccnc4)n3)c2)cc1]

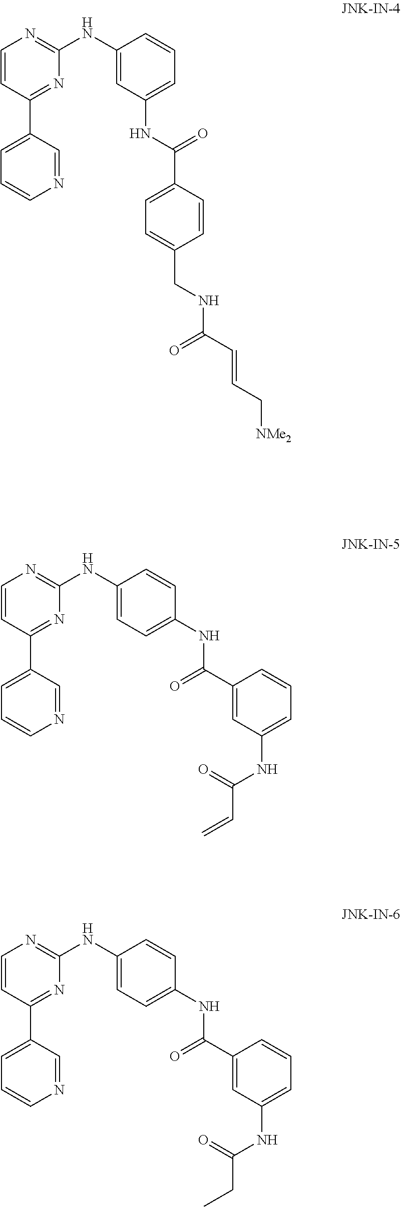 [SMILES: CN(C)C/C=C/C(=O)NCc1ccc(C(=O)Nc2cccc(Nc3nccc(-c4cccnc4)n3)c2)cc1 ; C=CC(=O)Nc1cccc(C(=O)Nc2ccc(Nc3nccc(-c4cccnc4)n3)cc2)c1 ; CCC(=O)Nc1cccc(C(=O)Nc2ccc(Nc3nccc(-c4cccnc4)n3)cc2)c1]

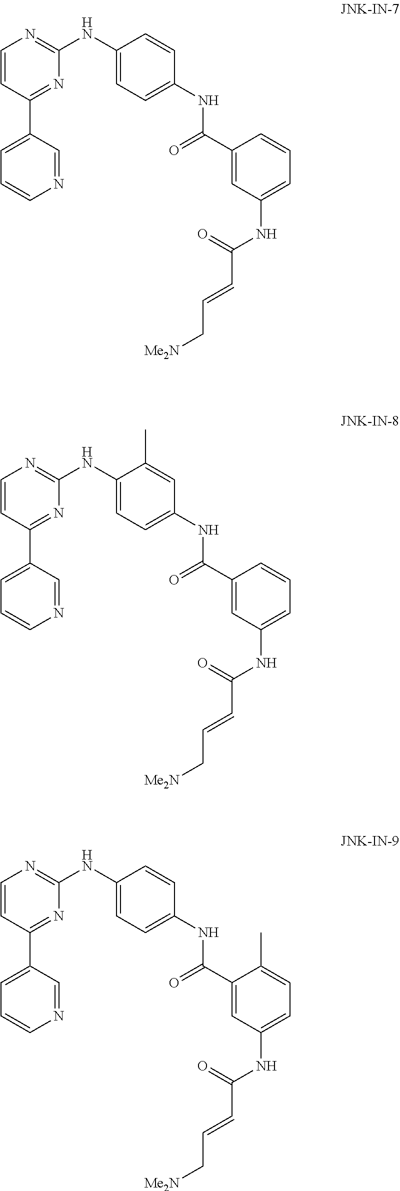 [SMILES: CN(C)C/C=C/C(=O)Nc1cccc(C(=O)Nc2ccc(Nc3nccc(-c4cccnc4)n3)cc2)c1 ; Cc1cc(NC(=O)c2cccc(NC(=O)/C=C/CN(C)C)c2)ccc1Nc1nccc(-c2cccnc2)n1 ; Cc1ccc(NC(=O)/C=C/CN(C)C)cc1C(=O)Nc1ccc(Nc2nccc(-c3cccnc3)n2)cc1]

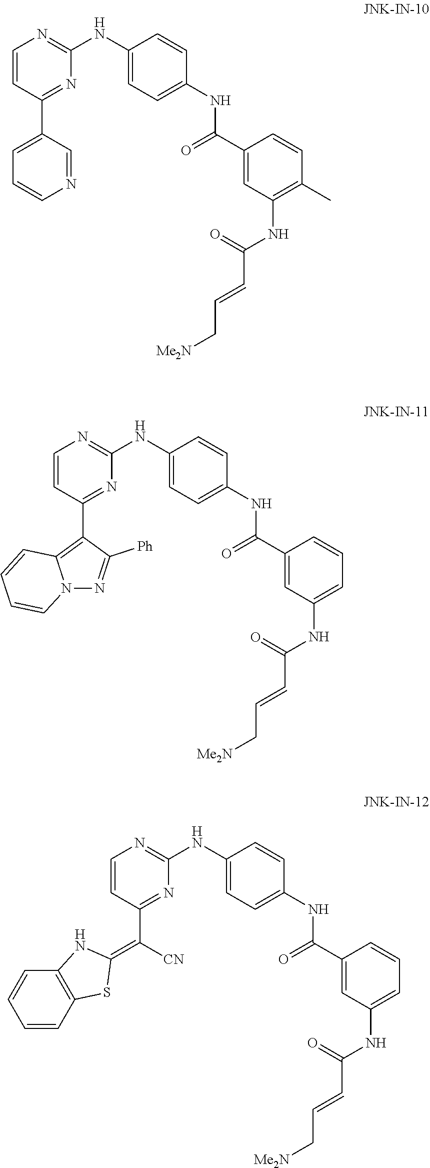 [SMILES: Cc1ccc(C(=O)Nc2ccc(Nc3nccc(-c4cccnc4)n3)cc2)cc1NC(=O)/C=C/CN(C)C ; CN(C)C/C=C/C(=O)Nc1cccc(C(=O)Nc2ccc(Nc3nccc(-c4c(-c5ccccc5)nn5ccccc45)n3)cc2)c1 ; CN(C)C/C=C/C(=O)Nc1cccc(C(=O)Nc2ccc(Nc3nccc(/C(C#N)=C4\Nc5ccccc5S4)n3)cc2)c1]

5. The culture system according to claim 1, wherein the basal medium comprises stemspan basal medium 1-100 ml; recombinant human stem cell factor (SCF) 10-500 ng/ml; Flt3 ligand (FL) 10-100 ng/ml, preferably 50-100 ng/ml; recombinant human thrombopoietin (TPO) 5-100 ng/ml, preferably 5-50 ng/ml; low-density lipoprotein (LDL) 2-10 μg/ml; and the JNK signaling pathway inhibitor is 0.5-10 uM, preferably 0.5-5 μM.

6. A method for expanding hematopoietic stem cells and/or hematopoietic progenitor cells, wherein hematopoietic stem cells and/or hematopoietic progenitor cells are cultured in vitro with the presence of a JNK signaling pathway inhibitor.

7. The method according to claim 6, wherein the JNK signaling pathway inhibitor is selected from one or more of the group consisting of JNK1 inhibitors, JNK2 inhibitors and JNK3 inhibitors.

8. The method according to claim 6, wherein the JNK signaling pathway inhibitor is selected from one or more of the group consisting of SP600125, AS601245 and AEG-3482.

9. The method according to claim 6, wherein the JNK signaling pathway inhibitor is any of the compounds represented by JNK-IN-1 to JNK-IN-12 as follows:

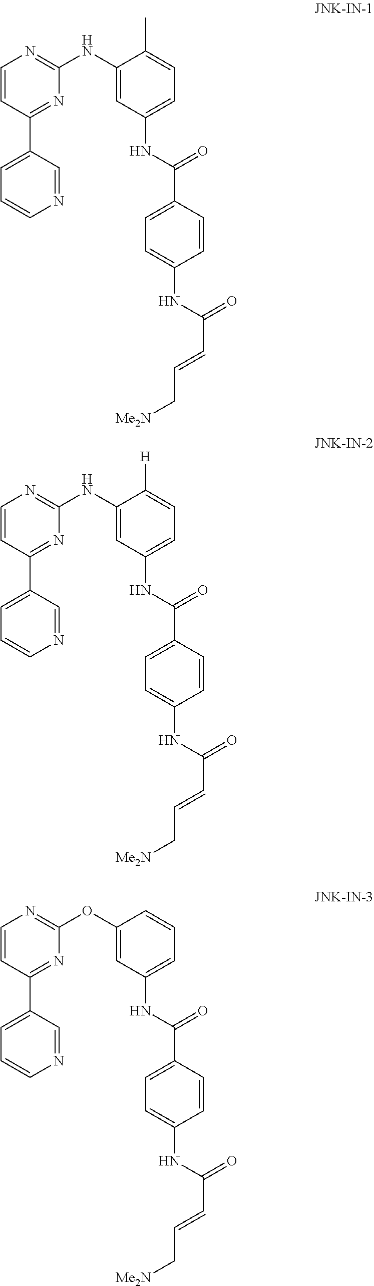

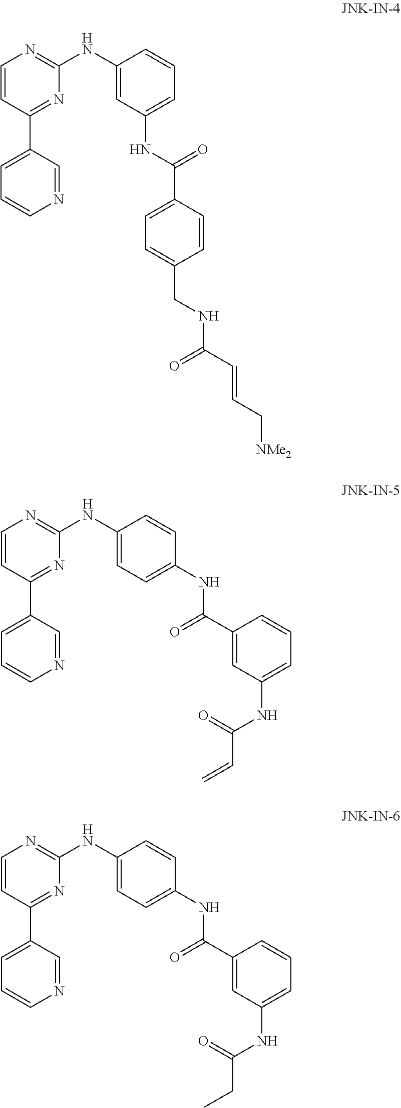 [SMILES: CN(C)C/C=C/C(=O)NCc1ccc(C(=O)Nc2cccc(Nc3nccc(-c4cccnc4)n3)c2)cc1 ; C=CC(=O)Nc1cccc(C(=O)Nc2ccc(Nc3nccc(-c4cccnc4)n3)cc2)c1 ; CCC(=O)Nc1cccc(C(=O)Nc2ccc(Nc3nccc(-c4cccnc4)n3)cc2)c1]

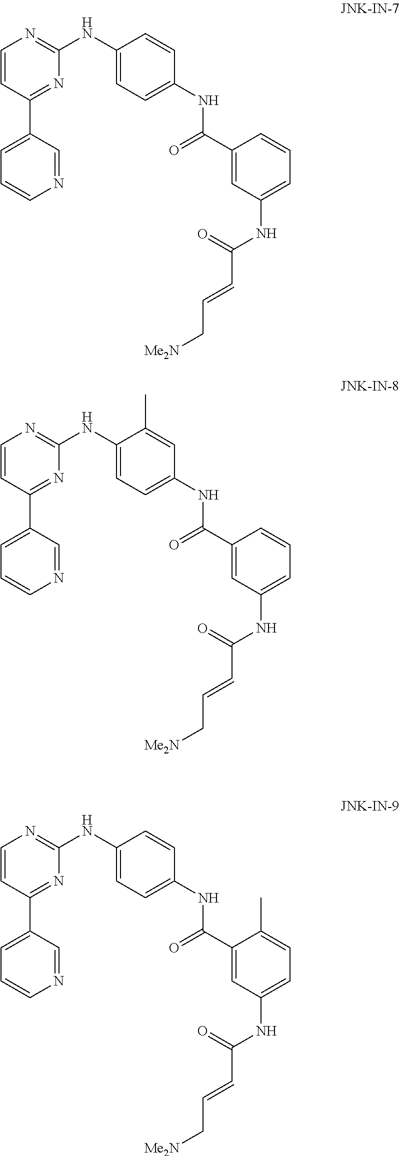 [SMILES: CN(C)C/C=C/C(=O)Nc1cccc(C(=O)Nc2ccc(Nc3nccc(-c4cccnc4)n3)cc2)c1 ; Cc1cc(NC(=O)c2cccc(NC(=O)/C=C/CN(C)C)c2)ccc1Nc1nccc(-c2cccnc2)n1 ; Cc1ccc(NC(=O)/C=C/CN(C)C)cc1C(=O)Nc1ccc(Nc2nccc(-c3cccnc3)n2)cc1]

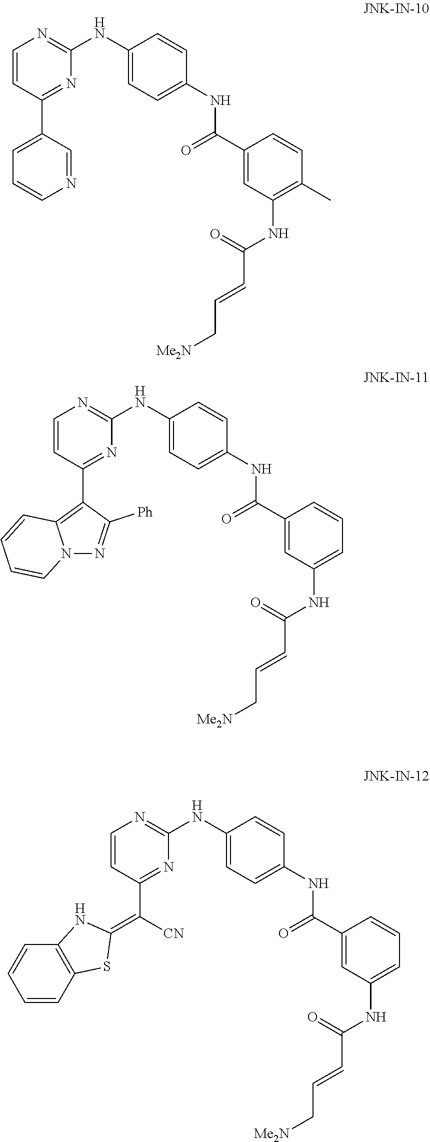 [SMILES: Cc1ccc(C(=O)Nc2ccc(Nc3nccc(-c4cccnc4)n3)cc2)cc1NC(=O)/C=C/CN(C)C ; CN(C)C/C=C/C(=O)Nc1cccc(C(=O)Nc2ccc(Nc3nccc(-c4c(-c5ccccc5)nn5ccccc45)n3)cc2)c1 ; CN(C)C/C=C/C(=O)Nc1cccc(C(=O)Nc2ccc(Nc3nccc(/C(C#N)=C4\Nc5ccccc5S4)n3)cc2)c1]

10. The method according to claim 6, wherein the method comprises the addition of stemspan basal medium, recombinant human stem cell factor, Flt3 ligand, recombinant human thrombopoietin and low-density lipoprotein.

11. The method according to claim 6, wherein the hematopoietic stem cells and/or hematopoietic progenitor cells are derived from bone marrow, liver, spleen, peripheral blood or cord blood.

12. At least one of hematopoietic stem cells and hematopoietic progenitor cells, wherein the hematopoietic stem cells and hematopoietic progenitor cells are obtained by using the culture system comprising a basal medium suitable for expanding stem cells; and a JNK signaling pathway inhibitor.

13. (canceled)

14. The at least one of hematopoietic stem cells and hematopoietic progenitor cells according to claim 12, wherein the JNK signaling pathway inhibitor is selected from one or more of the group consisting of JNK1 inhibitors, JNK2 inhibitors and JNK3 inhibitors.

15. The at least one of hematopoietic stem cells and hematopoietic progenitor cells according to claim 14, wherein the INK signaling pathway inhibitor is selected from one or more of the group consisting of SP600125, AS601245 and AEG-3482.

16. The at least one of hematopoietic stem cells and hematopoietic progenitor cells according to claim 15, wherein the INK signaling pathway inhibitor is any of the compounds represented by INK-IN-1 to INK-IN-12 as follows: 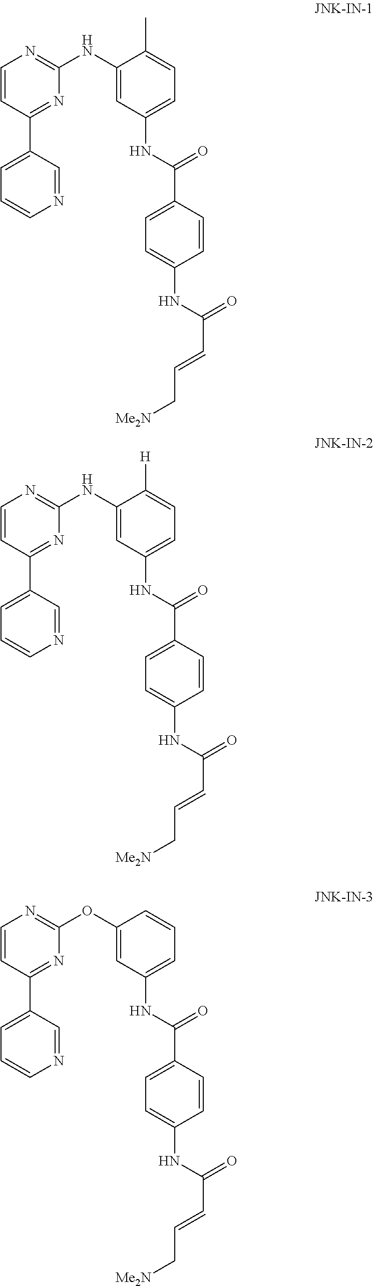 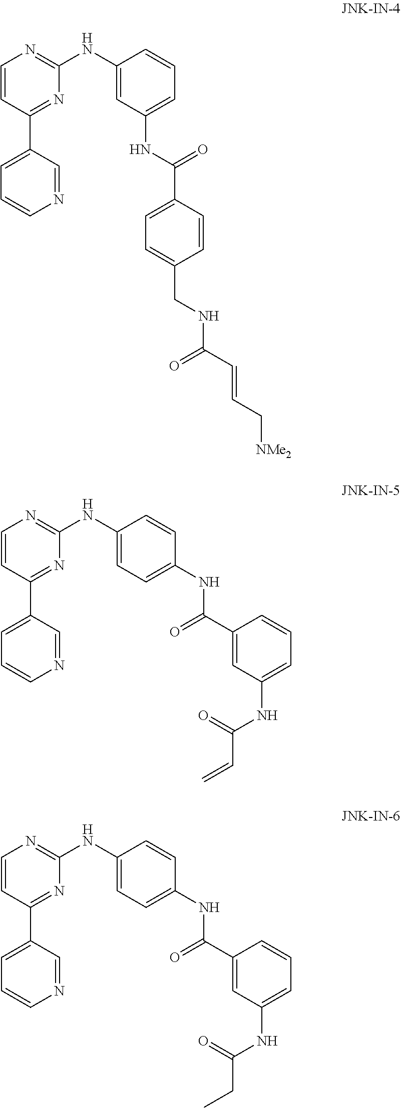 [SMILES: CN(C)C/C=C/C(=O)NCc1ccc(C(=O)Nc2cccc(Nc3nccc(-c4cccnc4)n3)c2)cc1 ; C=CC(=O)Nc1cccc(C(=O)Nc2ccc(Nc3nccc(-c4cccnc4)n3)cc2)c1 ; CCC(=O)Nc1cccc(C(=O)Nc2ccc(Nc3nccc(-c4cccnc4)n3)cc2)c1] 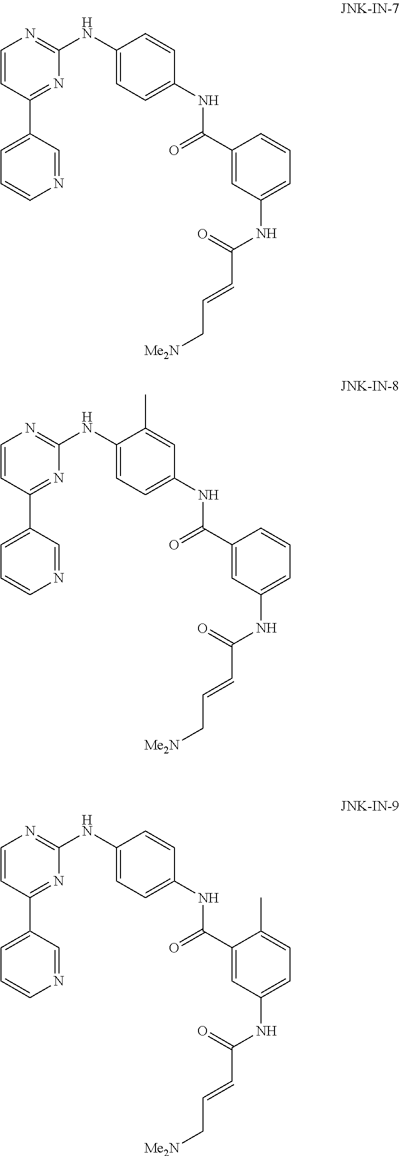 [SMILES: CN(C)C/C=C/C(=O)Nc1cccc(C(=O)Nc2ccc(Nc3nccc(-c4cccnc4)n3)cc2)c1 ; Cc1cc(NC(=O)c2cccc(NC(=O)/C=C/CN(C)C)c2)ccc1Nc1nccc(-c2cccnc2)n1 ; Cc1ccc(NC(=O)/C=C/CN(C)C)cc1C(=O)Nc1ccc(Nc2nccc(-c3cccnc3)n2)cc1] 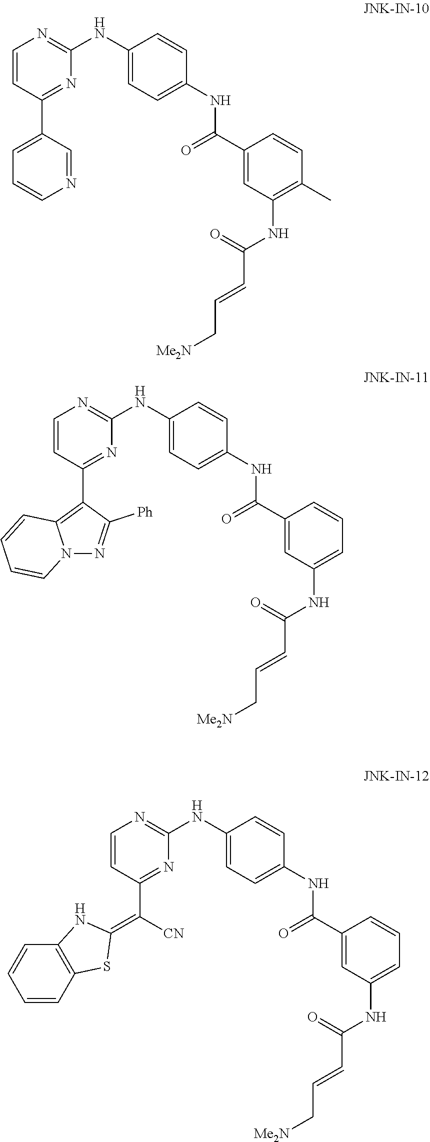 [SMILES: Cc1ccc(C(=O)Nc2ccc(Nc3nccc(-c4cccnc4)n3)cc2)cc1NC(=O)/C=C/CN(C)C ; CN(C)C/C=C/C(=O)Nc1cccc(C(=O)Nc2ccc(Nc3nccc(-c4c(-c5ccccc5)nn5ccccc45)n3)cc2)c1 ; CN(C)C/C=C/C(=O)Nc1cccc(C(=O)Nc2ccc(Nc3nccc(/C(C#N)=C4\Nc5ccccc5S4)n3)cc2)c1]
